(19) United States
(12) Patent Application Publication
(10) Pub. No.: US 2023/0201539 A1
(43) Pub. Date: Jun. 29, 2023

(54) U Guide for Scalpel Tip

(71) Applicant: Bard Access Systems, Inc., Salt Lake City, UT (US)

(72) Inventor: Glade H. Howell, Draper, UT (US)

(21) Appl. No.: 18/087,699

(22) Filed: Dec. 22, 2022

Related U.S. Application Data
(60) Provisional application No. 63/293,599, filed on Dec. 23, 2021.

(51) Int. Cl. A61M 25/09; A61B 17/3209

(52) U.S. Cl. CPC A61M 25/09041; A61B 17/32093; A61M 2025/09116; A61B 2017/320052

(57) ABSTRACT

A scalpel guide configured to be coupled with a scalpel blade. The scalpel guide engages a guidewire during use to constrain lateral displacement of the scalpel blade with respect to the guidewire. The scalpel guide includes laterally extending walls between which the guidewire is placed during use. The guide may be attached to the scalpel blade or formed integral to the scalpel blade.

### PRIORITY

This application claims the benefit of priority to U.S. Provisional Application No. 63/293,599, filed Dec. 23, 2021, which is incorporated by reference in its entirety into this application.

### BACKGROUND

Before placing a catheter in a blood vessel of a patient, it is common to nick the patient's skin about a needle tract at an insertion site for enlarging the needle tract to accommodate a catheter such as a central venous catheter. Typically, nicking the patient's skin is performed with a scalpel. It is common for a guidewire to be present within the needle track during enlarging process which adds difficulty since both the guidewire and the scalpel must be simultaneously inserted into the needle tract.

Disclosed herein are scalpel guides and methods of enlarging the needle tract that utilize the guidewire to facilitate positional control of the scalpel during the enlarging process.

### SUMMARY

Disclosed herein is scalpel guide according to some embodiments. The scalpel guide includes (i) a guidewire engagement portion extending along a first side of the scalpel guide, the guidewire engagement portion configured to constrain lateral displacement of the scalpel guide with respect to a guidewire and (ii) a blade coupling portion extending along a second side of the scalpel guide opposite the first side, the blade coupling portion configured to secure the scalpel guide to a scalpel blade, where, in use, the guide constrains a lateral displacement of the scalpel blade with respect to the guidewire.

The engagement portion may include a first laterally extending wall and a second laterally extending wall opposite the first laterally extending wall, and in use, the guidewire is disposed between the first laterally extending wall and the second laterally extending wall. The first laterally extending wall and/or the second lateral may include a curved portion configured to extend at least partially around the guidewire. In some embodiments, the engagement portion defines a “U” shape.

In some embodiments, the second laterally extending wall is disposed in longitudinal alignment with the first laterally extending wall and in other embodiments, the second laterally extending wall is disposed longitudinally offset from the first laterally extending wall.

In some embodiments, the engagement portion may include a third laterally extending wall disposed laterally opposite the first laterally extending wall or the second laterally extending wall.

In some embodiments, the engagement portion is configured to align the guidewire with a longitudinal axis of the scalpel guide.

In some embodiments, the blade coupling portion is configured to secure the scalpel guide to the scalpel blade along a back side edge of the scalpel blade, the back side edge disposed opposite a cutting edge of the scalpel blade.

In some embodiments, the longitudinal axis of the scalpel guide is disposed at an angle with respect to the cutting edge of the scalpel blade, and in some embodiments, the longitudinal axis of the scalpel guide is disposed parallel with a distal portion of the back side edge of the scalpel blade.

Also disclosed herein is a scalpel blade assembly that includes the scalpel guide of any embodiment described above coupled with a scalpel blade. The scalpel blade may be coupled with the scalpel guide via welding, a snap fit, over-molding, or the scalpel guide may be integrally formed with the scalpel blade.

Also disclosed herein is a scalpel that includes the scalpel blade assembly of any embodiment described above coupled with a scalpel handle.

In some embodiments, the longitudinal axis of the scalpel guide is disposed at an angle with respect to a longitudinal axis of the handle.

In some embodiments, includes a blade cover configured to extend over the scalpel blade assembly including the scalpel guide, and in some embodiments, the cover is selectively positionable between an extended position covering the scalpel blade assembly and a retracted position exposing the scalpel blade assembly.

Also disclosed herein is a method of enlarging a catheter insertion pathway. According to some embodiments, the method includes: (i) coupling a scalpel with a guidewire via a scalpel guide attached to a blade of the scalpel, where the guidewire is disposed within the insertion pathway, (ii) slidably displacing the scalpel guide along the guidewire so that a sharp point of the scalpel blade is disposed within the insertion pathway, and (iii) cutting a skin and/or a blood vessel wall via a cutting edge of the scalpel blade at a location opposite the guidewire to enlarge the insertion pathway. The scalpel guide is attached to the blade of the scalpel along a backside edge opposite the cutting edge of the scalpel blade, and the scalpel guide constrains a lateral displacement of the scalpel blade with respect to the guidewire.

In some embodiments of the method, the scalpel guide includes a first laterally extending wall and a second laterally extending wall opposite the first laterally extending wall, and coupling a scalpel with the guidewire includes placing the guidewire between the first laterally extending wall and the second laterally extending wall.

In some embodiments of the method, coupling a scalpel with a guidewire includes aligning the guidewire with a longitudinal axis of the scalpel guide, where in some embodiments, the longitudinal axis of the scalpel guide is disposed at an angle with respect to the cutting edge of the scalpel blade.

In some embodiments of the method, coupling a scalpel with a guidewire includes positioning a sharp point of the scalpel blade adjacent the guidewire and in some embodiments, the scalpel guide constrains the orientation of the scalpel blade so that the cutting edge faces away from the guidewire.

According to some embodiments of the method, the scalpel guide is: (i) integrally formed with the scalpel blade, (ii) welded to the scalpel blade, (iii) over-molded onto the scalpel blade, or (iv) attached to the scalpel blade via a snap fit.

These and other features of the concepts provided herein will become more apparent to those of skill in the art in view of the accompanying drawings and following description, which describe particular embodiments of such concepts in greater detail.

### DRAWINGS

FIG. 1A illustrates a scalpel including a scalpel guide in accordance with some embodiments.

FIG. 1B is a detailed view of the scalpel blade of FIG. 1A in accordance with some embodiments.

FIG. 1C is a cross-sectional end view the scalpel guide of FIG. 1A in accordance with some embodiments.

FIG. 2 illustrates the scalpel of FIGS. 1A-1C in use with a patient in accordance with some embodiments.

FIG. 3A is a detailed view of a second embodiment of the scalpel guide coupled with the scalpel blade in accordance with some embodiments.

FIG. 3B is a cross-sectional end view the scalpel guide of FIG. 3A in accordance with some embodiments.

FIG. 4A is a detailed view of a third embodiment of the scalpel guide over-molded onto the scalpel blade in accordance with some embodiments.

FIG. 4B is a cross-sectional end view the scalpel guide of FIG. 4A in accordance with some embodiments.

FIG. 5A is a detailed view of a fourth embodiment of the scalpel guide integrally formed with the scalpel blade in accordance with some embodiments.

FIG. 5B is a cross-sectional end view the scalpel guide of FIG. 5A in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a scalpel includes a portion or section of the scalpel intended to be near a clinician when the scalpel is used on a patient. Likewise, a “proximal length” of, for example, the scalpel includes a length of the scalpel intended to be near the clinician when the scalpel is used on the patient. A “proximal end” of, for example, the scalpel includes an end of the scalpel intended to be near the clinician when the scalpel is used on the patient. The proximal portion, the proximal section, or the proximal length of the scalpel can include the proximal end of the scalpel; however, the proximal portion, the proximal section, or the proximal length of the scalpel need not include the proximal end of the scalpel. That is, unless context suggests otherwise, the proximal portion, the proximal section, or the proximal length of the scalpel is not a terminal portion or terminal length of the scalpel.

With respect to “distal,” a “distal portion” or a “distal section” of, for example, a scalpel includes a portion or section of the scalpel intended to be near or in a patient when the scalpel is used on the patient. Likewise, a “distal length” of, for example, the scalpel includes a length of the scalpel intended to be near or in the patient when the scalpel is used on the patient. A “distal end” of, for example, the scalpel includes an end of the scalpel intended to be near or in the patient when the scalpel is used on the patient. The distal portion, the distal section, or the distal length of the scalpel can include the distal end of the scalpel; however, the distal portion, the distal section, or the distal length of the scalpel need not include the distal end of the scalpel. That is, unless context suggests otherwise, the distal portion, the distal section, or the distal length of the scalpel is not a terminal portion or terminal length of the scalpel.

Unless defined otherwise, all technical and scientific terms used herein have the same meaning as commonly understood by those of ordinary skill in the art.

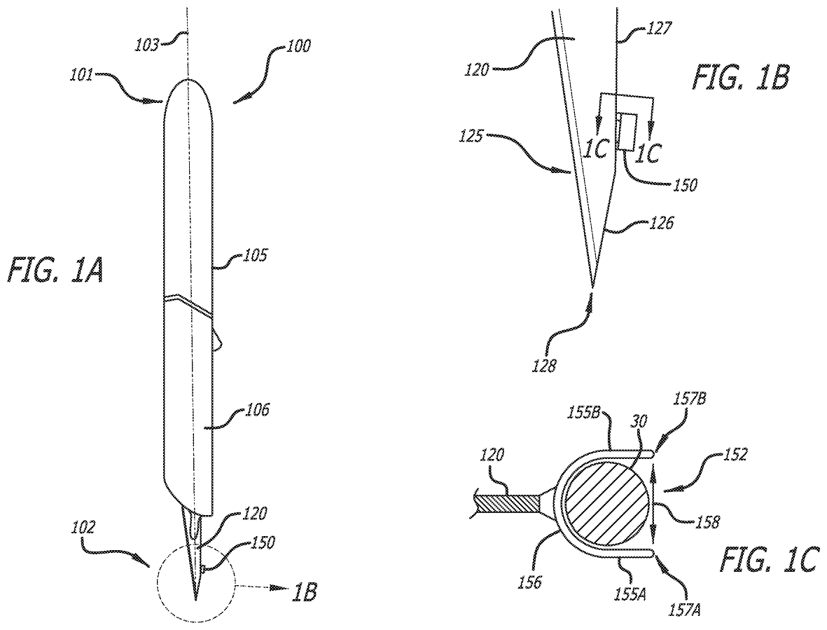

FIGS. 1A-1C illustrate a first scalpel 100 having a guide 150 in accordance with some embodiments. FIG. 1A illustrates a side view of the scalpel 100. FIG. 1B is a detail view of a distal tip portion of a blade 120 of the scalpel 100 and FIG. 1C is a detailed cross-sectional view of a portion of the blade 120 cut along sectioning lines 1C-1C providing an end view of the guide 150 attached to the blade 120.

With reference to the FIG. 1A, the scalpel 100 general includes a handle 105 extending between a proximal end 101 and the blade 120 attached thereto at the distal end 102. The blade 120 may be permanently attached to the handle 105 or alternatively, the blade 120 may be selectively attachable to and detachable from the handle 105. For illustration purposes, the scalpel 100 may define a longitudinal axis 103.

In some embodiments, the scalpel 100 may include a blade cover 106 as a safety precaution that extends around and distally beyond the blade 120. In some embodiments, the blade cover 106 may be removable from the scalpel 100 for use of the blade 120. In other embodiments, the blade cover 106 may be positionally attached to the handle 105 so that the blade cover 106 may be selectively disposed between a “use” position exposing the blade 120 and a “safety” position covering the blade 120. In some embodiments, positioning the blade cover 106 includes longitudinally sliding the blade cover 106 along the handle 105.

With reference to FIG. 1B, the blade 120 includes a cutting edge 125. A distal back side edge 126 is disposed opposite the cutting edge 125 and converges with the cutting edge 125 to form the sharp point 128 at the distal most tip of the blade 120. A proximal back side edge 127 extends proximally away from a junction with the distal back side edge 126. The guide 150 is attached to the blade 120 along either of the distal back side edge 126 or the proximal back side edge 127 or both. The guide 150 may be attached to the blade 120 via any suitable attachment method such as welding, adhesive boding, or snap-fitting, for example.

With reference to FIG. 1C, according to a first embodiment, the guide 150 includes a first wall 155A extending laterally away from the blade 120 and a second wall 155B also extending laterally away from the blade 120 defining a receiving space 152 between the first and second walls 155A, 155B, where the receiving space 152 is configured to receive a guidewire 30 therein as shown. The receiving space 152 is sized to accept a guidewire therein as further described below. A back wall 156 extends between the first and second walls 155A, 155B adjacent the blade 120. In some embodiments, the first and second walls 155A, 155B are coupled to each other to form the back wall 156 defining a “U” shape, i.e., the back wall 156 defines a full radius between the first and second walls 155A, 155B. Although not required, the guide 150 may be attached to the blade 120 so that the blade 150 bisects the receiving space 152. Similarly, although not required, the first and second walls 155A, 155B may be in parallel with the blade 120 and/or in parallel with each other.

The receiving space 152 is sized so that the guidewire 30 is laterally constrained between the first and second walls 155A, 155B. In use, the first and second walls 155A, 155B inhibit lateral displacement of the guidewire 30 with respect to the blade 120 and vice versa. The receiving space 152 is sized to enable longitudinal displacement of the guidewire 30 with respect to the guide 150, such as longitudinal sliding displacement.

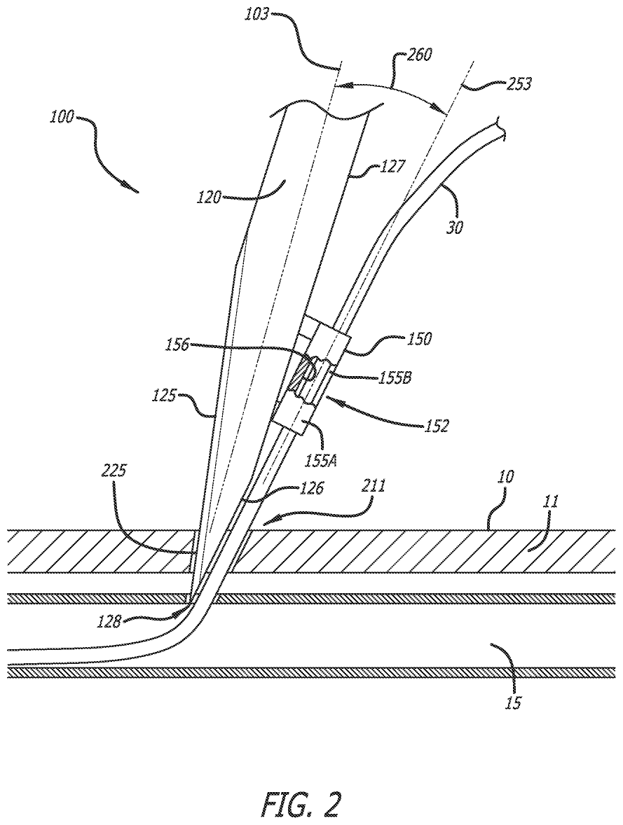

In some embodiments, although not required, the back wall 156 may be positioned with respect to the distal back side edge 126 so that the back wall 156 is aligned with the distal back side edge 126. In other words, when the guidewire 30 is positioned within the receiving space 152 so that the guidewire 30 is parallel with the back wall 156, the guidewire 30 may also extend distally away from the guide 150 in a direction parallel with the distal back side edge 126 (see FIG. 2).

In some embodiments, although not required, the first and second walls 155A, 155B may be figured to deflect during use. For example, a separation distance 158 between the endpoints (or end portions) 157A, 157B of the first and second walls 155A, 155B, may be less than a diameter of the guidewire 30. According to such embodiments, the guidewire 30 may be inhibited from laterally displacing out of the receiving space 152 without deflecting the endpoints 157A, 157B outward. During use of such an embodiment, the guidewire 30 may be laterally displaced into the receiving space 152 causing the endpoints 157A, 157B to deflect outward. Similarly, the guidewire 30 may be laterally displaced out of the receiving space 152 also causing the endpoints 157A, 157B to deflect outward.

FIG. 2 illustrates a side view of a distal portion of the scalpel 100 in use with a patient 10. As shown via a cut-away portion of the guide 150, the guidewire 30 is positioned within the receiving space 152 so that the guidewire 30 is constrained by the first and second walls 155A, 155B and the back wall 156. As shown, the guidewire is parallel with the back wall 156 and the distal back side edge 126. As also shown, the guidewire 30 diverges away from the longitudinal axis 103 of the scalpel 100 according to an angle 260 as the guidewire 30 proximally extends away from the guide 150. As also shown, the guide 150 constrains the orientation of the blade 120 so that the cutting edge 125 faces away from the guidewire 30.

As further illustrated in FIG. 2, the guidewire 30 is inserted through the skin 11 of the patient 10 and into a blood vessel 15 along an insertion pathway 211 (e.g., a needle tract). The blade 120 has been distally displaced along the guidewire 30 as laterally constrained by the guide 150 until the sharp point 128 is disposed within the blood vessel 15. As illustrated, the cutting edge 125 has defined a nick 225 (i.e., a cut portion of the skin and/or blood vessel wall) to enlarge the insertion pathway 211.

Methods include methods of using the scalpel 100 or more generally a methods of enlarging an insertion pathway for a catheter. Typically, prior to using the scalpel or enlarging the insertion pathway, access to the blood vessel includes placement of a guidewire along an insertion pathway extending through the skin and into the blood vessel. The clinician may couple the scalpel with the guidewire via the guide. More specifically the clinician may place the guidewire within the receiving portion of the guide so that lateral displacement of the blade of the scalpel is constrained by the guidewire. The clinician may couple the scalpel with the guidewire while the sharp point of the blade is disposed above the skin. The clinician may then distally slide the blade along the guidewire as constrained by the guide toward the skin. The clinician may further distally slide the blade along the guidewire to insert the sharp point into the patient through the insertion pathway and along the guidewire. During further insertion, the cutting edge of the blade engages the skin and nicks or cuts the skin in the process thereby enlarging the insertion pathway. The clinician may continue to insert the sharp point so that the sharp point enters the blood vessel and so that the cutting edge of the blade engages the blood vessel wall to nick the blood vessel wall and enlarge the portion of the insertion pathway extending through the blood vessel wall. After the insertion pathway is sufficiently enlarged, the clinician may proximally retract the blade from the patient and separate the scalpel from the guidewire. In some embodiments, a method may include attaching the guide to the blade of the scalpel. In further embodiments, the clinician may proximally displace a cover of the scalpel to expose the blade including the guide. The clinician may also distally displace the cover of the scalpel to extend over the blade including the guide.

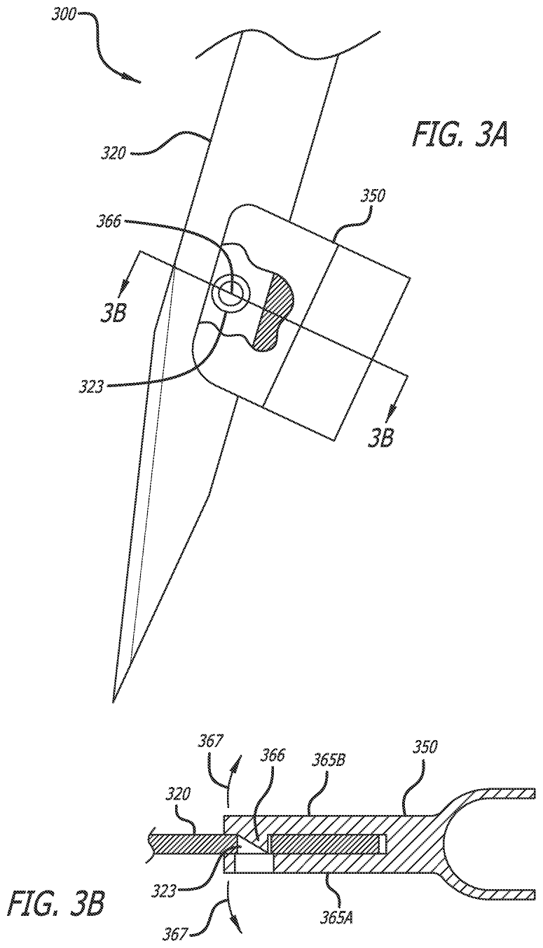

FIGS. 3A and 3B illustrate a second embodiment of a scalpel 300 that can, in certain respects, resemble components of the scalpel 100 described in connection with FIGS. 1A-2. It will be appreciated that all the illustrated embodiments may have analogous features. Accordingly, like features are designated with like reference numerals, having a leading digit of “3.” For instance, the blade is designated as “120” in FIGS. 1A-2, and an analogous blade is designated as “320” in FIGS. 3A-3B. Relevant disclosure set forth above regarding similarly identified features thus may not be repeated hereafter. Moreover, specific features of the scalpel 100 and related components shown in FIGS. 1A-2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the scalpel of FIGS. 3A-3B. Any suitable combination of the features, and variations of the same, described with respect to the scalpel 100 and components illustrated in FIGS. 1A-2 can be employed with the scalpel and components of FIGS. 3A-3B, and vice versa. This pattern of disclosure applies equally to further embodiments depicted in subsequent figures and described hereafter.

The scalpel 300 includes a guide 350 configured to couple with the blade 320 via a snap fit in accordance with some embodiments. FIG. 3A illustrates a detail view of a distal tip portion of the blade 320 of the scalpel 300 and FIG. 3B is a detailed cross-sectional view of a portion of the blade 120 cut along sectioning lines 3B-3B providing an end view of the guide 350 attached to the blade 320. The opposing wall members 365A, 365B define a slot therebetween that is sized to receive the blade 320 therein. An aperture 323 extending through the blade 320 is configured to receive a protrusion 366 extending at least partially therethrough. In the assembled state, the blade 320 is disposed in the slot (i.e., between the opposing wall members 365A, 365B) and the protrusion 366 is disposed within the aperture 323 thereby securing the guide 350 to the blade 320. In some embodiments, the guide 350 may be formed of a plastic material via the injection molding process.

The opposing wall members 365A, 365B are configured to deflect away from each other as indicated by the arrows 367 so that the blade 320 may be inserted within the slot. After insertion of the blade 320, the opposing wall members 365A, 365B may self-deflect toward each other to displace the protrusion 366 within the aperture 323, thereby securing the guide 350 to the blade 320.

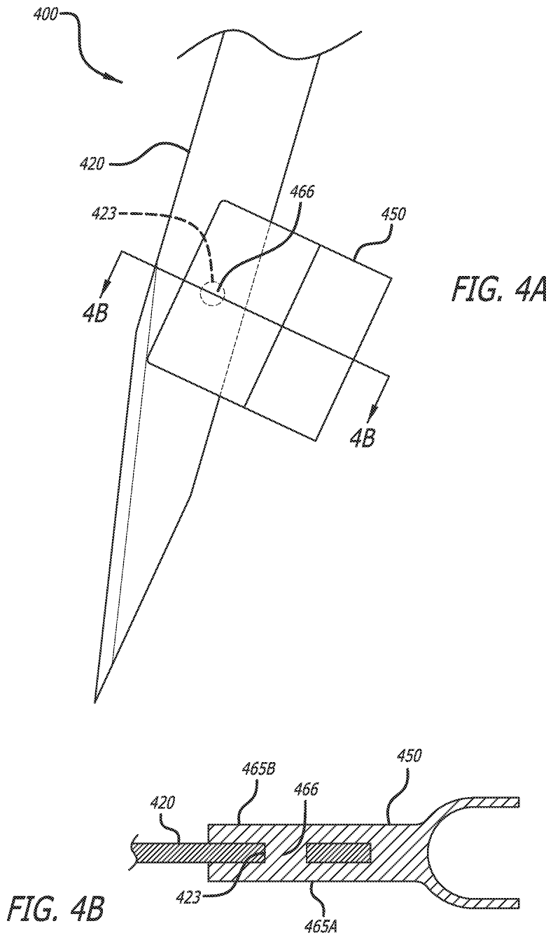

FIGS. 4A-4B illustrate a third embodiment of the scalpel. The scalpel 400 includes a guide 450 over-molded onto the blade 420 via the plastic injection molding process in accordance with some embodiments. FIG. 4A illustrates a detail view of a distal tip portion of the blade 420 of the scalpel 400 and FIG. 4B is a detailed cross-sectional view of a portion of the blade 120 cut along sectioning lines 4B-4B providing an end view of the guide 450 attached to the blade 420. The opposing wall members 465A, 465B define a slot therebetween that is sized to receive the blade 420 therein. An aperture 423 extending through the blade 420 is configured to receive a connecting portion 466 extending through the aperture 423. In the assembled state (i.e., when the guide 450 is over molded onto the blade 420), the blade 420 is disposed in the slot (i.e., between the opposing wall members 465A, 465B) and the connecting portion 466 is molded through the aperture 423 thereby securing the guide 450 to the blade 420.

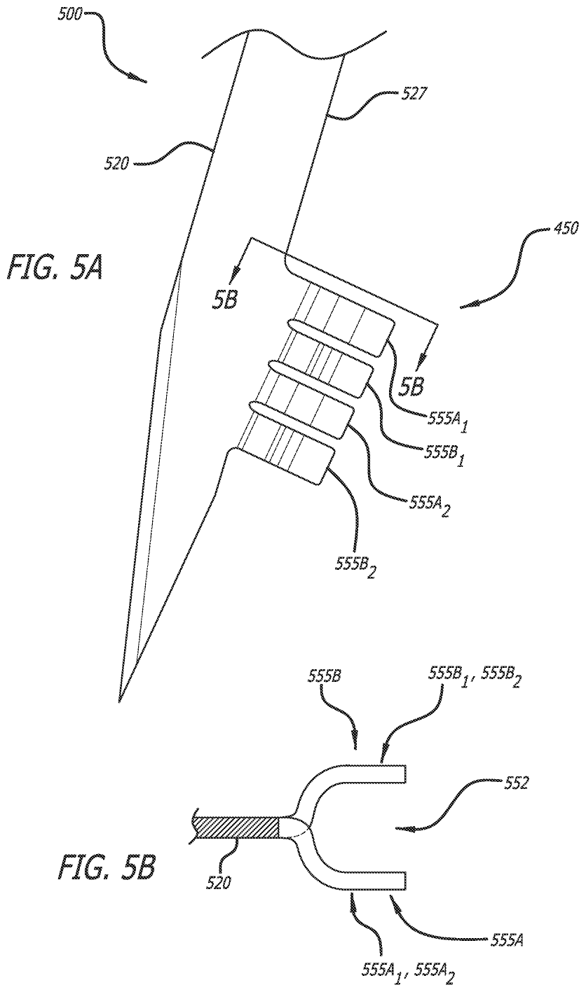

FIGS. 5A and 5B illustrate a fourth embodiment of the scalpel. The scalpel 500 includes a guide 550 that is formed integral with the blade 520 in accordance with some embodiments. FIG. 5A illustrates a detail view of a distal tip portion of the blade 520 of the scalpel 500 and FIG. 5B is a detailed cross-sectional view of a portion of the blade 520 cut along sectioning lines 5B-5B providing an end view of the guide 550 integral to the blade 520. The guide 550 includes a series of tabs 555A₁, 555B₁, 555A₂, and 555B₂ extending away from the proximal back edge 527. The tabs 555A₁, 555B₁, 555A₂, and 555B₂ are longitudinally offset from each other along the blade 520. In some embodiments, the tabs 555A₁, 555B₁, 555A₂, and 555B₂ may be arranged in an alternating fashion as illustrated. The tabs 555A₁, 555B₁, 555A₂, and 555B₂ are formed to define the first and second walls 555A, 555B. The first and second walls 555A, 555B define the receiving space 552 and define a functionality that is similar to the first and second walls 155A, 155B of the guide 150 of FIGS. 1A-2. Although in the illustrated embodiment, the guide 550 include four tabs, in other embodiments, the guide 550 may include two, three, five, or more tabs.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A scalpel guide, comprising:
a blade coupling portion extending along a first side of the scalpel guide opposite the first side, the blade coupling portion configured to secure the scalpel guide to a scalpel blade; and
a guidewire engagement portion extending along a second side of the scalpel guide opposite the first side, the guidewire engagement portion configured to constrain lateral displacement of the scalpel guide with respect to a guidewire, wherein in use the guide constrains lateral displacement of the scalpel blade with respect to the guidewire.

2. The scalpel guide of claim 1, wherein:
the engagement portion includes a first laterally extending wall and a second laterally extending wall opposite the first laterally extending wall, and
in use, the guidewire is disposed between the first laterally extending wall and second laterally extending wall.

3. The scalpel guide of claim 2, wherein the first laterally extending wall and/or the second laterally extending wall include a curved portion configured to extend at least partially around the guidewire.

4. The scalpel guide of claim 2, wherein the second laterally extending wall is disposed in longitudinal alignment with the first laterally extending wall.

5. The scalpel guide of claim 2, wherein the second laterally extending wall is disposed longitudinally offset from the first laterally extending wall.

6. The scalpel guide of claim 2, wherein the engagement portion further includes a third laterally extending wall disposed laterally opposite the first laterally extending wall or the second laterally extending wall.

7. The scalpel guide of claim 1, wherein the engagement portion is configured to align a longitudinal axis of the scalpel guide with the guidewire.

8. The scalpel guide of claim 1, wherein engagement portion defines a “U” shape.

9. The scalpel guide of claim 1, wherein the blade coupling portion is configured to secure the scalpel guide to the scalpel blade along a back side edge of the scalpel blade, the back side edge disposed opposite a cutting edge of the scalpel blade.

10. The scalpel guide of claim 9, wherein the longitudinal axis of the scalpel guide is disposed at an angle with respect to the cutting edge of the scalpel blade.

11. The scalpel guide of claim 9, wherein the longitudinal axis of the scalpel guide is disposed parallel with a distal portion of the back side edge of the scalpel blade.

12. A scalpel blade assembly, comprising:
the scalpel guide of claim 1; and
the scalpel blade of claim 1 coupled with the scalpel guide.

13. The scalpel blade assembly of claim 12, wherein the scalpel guide is integrally formed with the scalpel blade.

14. The scalpel blade assembly of claim 12, wherein the scalpel guide is welded to the scalpel blade.

15. The scalpel blade assembly of claim 12, wherein the scalpel guide is over-molded onto the scalpel blade.

16. The scalpel blade assembly of claim 12, wherein the scalpel guide is attached to the scalpel blade via a snap fit.

17. A scalpel, comprising:
the scalpel blade assembly of claim 12; and
a scalpel handle coupled with the scalpel blade assembly.

18. The scalpel of claim 17, wherein the longitudinal axis of the scalpel guide is disposed at an angle with respect to a longitudinal axis of the scalpel handle.

19. The scalpel of claim 18, further comprising a blade cover configured to extend over the scalpel assembly including the scalpel guide.

20. The scalpel of claim 19, wherein the cover is selectively positionable between:
an extended position covering the scalpel blade assembly, and
a retracted position exposing the scalpel blade assembly.

21-30. (canceled)
